(19) United States
(12) Patent Application Publication
(10) Pub. No.: US 2025/0049056 A1
(43) Pub. Date: Feb. 13, 2025

(54) METHOD FOR TREATING MILK OR A PLANT-BASED BEVERAGE AND DEVICE IMPLEMENTING SAID TREATMENT METHOD

(71) Applicant: DE' LONGHI APPLIANCES S.r.l., Treviso (IT)

(72) Inventors: Antonio PANCIERA, Treviso (IT); Alessandro BENEDETTI, Treviso (IT); Simone ROSETTA, Treviso (IT)

(73) Assignee: DE' LONGHI APPLIANCES S.r.l., Treviso (IT)

(21) Appl. No.: 18/719,708

(22) PCT Filed: Dec. 16, 2022
(86) PCT No.: PCT/EP2022/086283

§ 371 Date: Jun. 13, 2024

(30) Foreign Application Priority Data
Dec. 23, 2021 (IT) 102021000032594

(51) Int. Cl. A23C 9/00; G01J 3/00; G01N 33/14

(52) U.S. Cl. CPC A23C 9/00; G01J 3/00; G01N 33/14

(57) ABSTRACT

The method for treating milk or a plant-based beverage comprises creating a database containing associations between combinations of values of content by weight of proteins and carbohydrates of the milk or plant-based beverage to be treated and corresponding values of at least one treatment parameter for the milk or plant-based beverage; acquiring the content by weight of proteins and carbohydrates of the milk or plant-based beverage to be treated; and treating the milk or plant-based beverage with the value of the at least one treatment parameter corresponding in the database to the acquired values of the content by weight of proteins and carbohydrates.

### CROSS-REFERENCE TO RELATED APPLICATIONS

This is a U.S. National Phase Application under 35 U.S.C. 371 of International Patent Application No. PCT/EP2022/086283, filed Dec. 16, 2022, which claims benefit of priority to Italian Patent Application No. 102021000032594, filed Dec. 23, 2021. The entire contents of these applications are hereby incorporated by reference.

### FIELD OF THE INVENTION

The present invention relates to a method for treating milk or a plant-based beverage, and to a device, particularly but not necessarily a coffee machine, which implements said treatment method.

### BACKGROUND

It is well known that many models of automatic coffee machines are also capable of frothing and heating milk, for example to make a cappuccino.

The result in the cup largely depends on the physicochemical properties of the milk used.

As is well known, today various types of milk of animal or plant origin can be used to produce a beverage according to consumer tastes.

In general, the known coffee machines are set with standard parameters for frothing and heating that do not take into account, therefore, the type of milk used, with the inevitable consequence that the result in the cup is extremely variable and does not always meet demands.

EP3332678 A1 describes a spectrometric sensor used only to discriminate whether the milk has deteriorated and is thus no longer fit to be consumed.

### SUMMARY

The technical task of the present invention, therefore, is to provide a method for treating milk or a plant-based beverage that enables the aforementioned technical drawbacks of the prior art to be eliminated.

Within the scope of this technical task, one object of the invention is to treat a milk or a plant-based beverage with predictable, repeatable results, irrespective of the type of milk or plant-based beverage treated.

Another object of the invention is to obtain, in the cup, a milk beverage or plant-based beverage with improved organoleptic properties, consistency and amount of froth, irrespective of the type of milk or plant-based beverage treated.

Another object of the invention is to provide a method for treating milk or a plant-based beverage that can be carried out automatically without any specific knowledge or abilities being required of the operator.

The technical task, as well as these and other objects, are achieved according to the present invention by providing a method for treating milk or a plant-based beverage, characterized in that it comprises the following steps:
creating a database containing associations between combinations of values of content by weight of proteins and carbohydrates of the milk or plant-based beverage to be treated and corresponding values of at least one treatment parameter for the milk or plant-based beverage;
acquiring the content by weight of proteins and carbohydrates of the milk or plant-based beverage to be treated; and
treating the milk or plant-based beverage with the value of said at least one treatment parameter corresponding in the database to the acquired values of the content by weight of proteins and carbohydrates.

In one embodiment of the invention, said acquisition step includes a spectrophotometric determination performed directly on said milk or plant-based beverage to be treated.

In a further embodiment of the invention, said acquisition step includes an automatic optical reading of information provided on a package of said milk or plant-based beverage to be treated. In a further embodiment of the invention, said acquisition step includes a personal acquisition of information.

The method in accordance with the invention makes it possible to obtain predictable and repeatable results when the type of milk or plant-based beverage used changes.

In particular, the milk for preparing a beverage can be of animal origin—whole, skimmed or partly skimmed—or plant origin, for example, from soy, rice, almond, oats, coconut, etc.

The milk or plant-based beverage to be treated is identified through a determination of the content by weight of proteins and carbohydrates and the treatment parameters for the milk or plant-based beverage are modulated according to the data determined so as to obtain in the cup substantially the same result when the type of milk or plant-based beverage used changes.

Optimal frothing and/or heating can be achieved almost automatically without relying on any specific experience of the consumer, who is required only to press a pushbutton on the control panel of a coffee machine or a milk frothing device.

In one embodiment of the invention, said database comprises a first association, wherein a first value of said at least one treatment parameter corresponds to a content by weight of proteins no lower than a first threshold value and a content by weight of carbohydrates no higher than a second threshold value.

In one embodiment of the invention, said database comprises a second association, wherein a second value of said at least one treatment parameter corresponds to a content by weight of proteins lower than said first threshold value and to a content by weight of carbohydrates higher than said second threshold value.

In one embodiment of the invention, said database comprises a third association, wherein a third value of said at least one treatment parameter corresponds to a content by weight of proteins higher than said first threshold value and to a content by weight of carbohydrates higher than said second threshold value, or to a content by weight of proteins lower than said first threshold value and to a content by weight of carbohydrates lower than said second threshold value.

In one embodiment of the invention, said at least one treatment parameter comprises at least the amount of thermal energy to be transferred overall and/or in the unit of time to the milk or plant-based beverage to be treated.

In one embodiment of the invention, said at least one treatment parameter further comprises at least the amount of air to be transferred overall and/or in the unit of time to the milk or plant-based beverage to be treated.

The present invention also discloses a device, particularly a coffee machine or a milk frothing device, comprising a receptacle for a milk or plant-based beverage to be treated, a station for said receptacle, a means for heating the milk or plant-based beverage, and a means for frothing the milk or plant-based beverage, characterized in that it has a spectrophotometer configured to determine the content by weight of proteins and carbohydrates of the milk or plant-based beverage to be treated present in said receptacle, and in that it has an electronic controller having in its memory a database containing associations between combinations of values of content by weight of proteins and carbohydrates of the milk or plant-based beverage to be treated and corresponding values of at least one treatment parameter for the milk or plant-based beverage, said controller being configured to control the treatment of the milk or plant-based beverage with the value of said at least one treatment parameter corresponding, in the database, to the determined values of the content by weight of proteins and carbohydrates.

Preferably, the device also has a temperature sensor for sensing the temperature of the milk or plant-based beverage to be treated present in said receptacle.

### BRIEF DESCRIPTION OF THE DRAWINGS

Further features and advantages of the invention will more fully emerge from the description of a preferred, but not exclusive, embodiment thereof, of the method for treating milk or a plant-based beverage according to the invention by way of non-limiting example in the drawings, in which:

FIG. 1 shows a first embodiment of a coffee machine suitable for implementing the method of the invention;

FIG. 2 shows a second embodiment of a coffee machine suitable for implementing the method of the invention; and

FIG. 3 shows a graph of the values determined by the spectrophotometer when the types of milk used change.

### DETAILED DESCRIPTION

With reference to the aforesaid figures, they show an automatic device, particularly a coffee machine, denoted in its entirety by the reference number 1, and specifically configured to treat a milk or a plant-based beverage.

The coffee machine 1 is of the type generically comprising a water circuit (not shown) having, in a cascade arrangement, a water reservoir or a connection to a water main, a water supply pump, a boiler for heating the water, a coffee brewing unit, and an external dispenser of the coffee extract.

The coffee machine 1 further comprises a station 2 for a receptacle 3 for containing milk or a plant-based beverage, a means 4 for heating the milk or plant-based beverage and a means 5 for frothing the milk or plant-based beverage.

The heating means 4 can be of the steam type or an electric heating element.

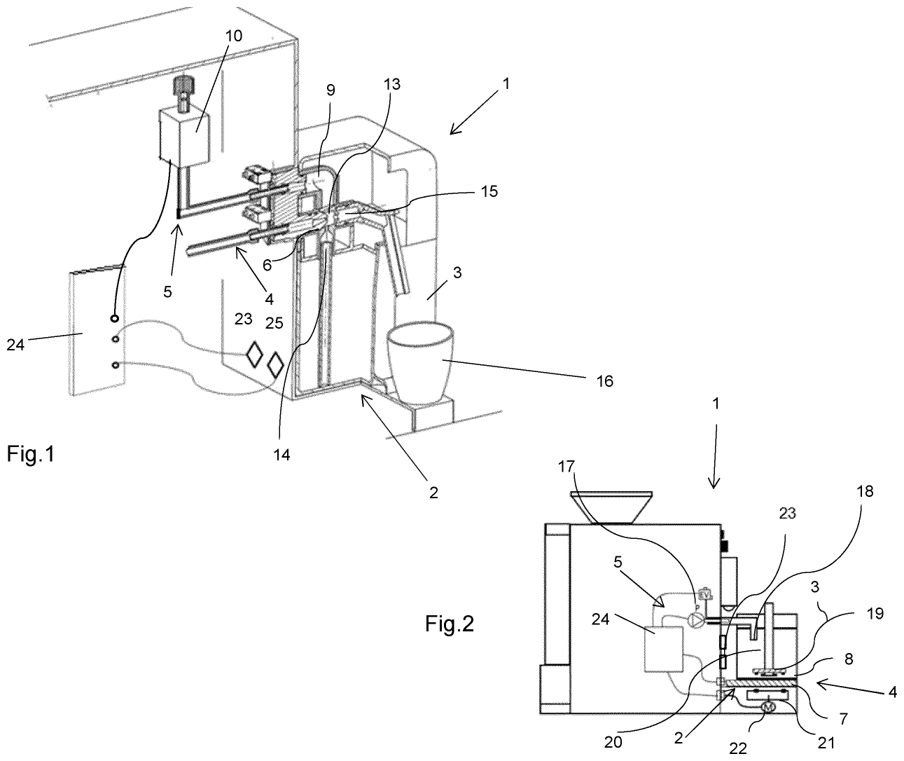

With reference to the solution shown in FIG. 1, the heating means 4 includes a steam dispenser 6 associated with a source of steam (not shown), for example a boiler supplied by a water pump.

Again with reference to the solution shown in FIG. 1, the frothing means 5 includes an air conduit 9 fitted with a progressively opening valve 10. The air conduit 9 is connected to a Venturi effect mixing chamber 13. Also connected to the mixing chamber 13 there is a conduit 14 for the milk or plant-based beverage, the conduit extending inside the receptacle 3 and the steam dispenser 6, which, by dispensing a flow of steam, triggers the Venturi effect whereby air and the milk or plant-based beverage are drawn into the mixing chamber 13. The mixing chamber 13 is also connected to a conduit 15 for dispensing into a cup 16.

With reference to the solution shown in FIG. 2, by contrast, the heating means 4 includes an electric heater 7, for example incorporated in a base 8 of the station 2 for the receptacle 3.

Again with reference to the solution shown in FIG. 2, the frothing means 5 includes an air pump 17 having a dispensing conduit 18 which extends inside the receptacle 3, and a mechanical stirrer 19 present on the bottom of the receptacle 3. The mechanical stirrer 19 is supported in free rotation at the lower end of a vertical tube 20 supported by the receptacle 3. The vertical tube 20 has an upper end that projects from the receptacle 3 and bends downwards to dispense into a cup. The receptacle 3 is hermetically sealed by a removable lid so that the air pressure generated by the air pump 17 inside the receptacle 3 can push the milk or plant-based beverage upwards through the vertical tube 20 in order to be dispensed into a cup. The actuation of the mechanical stirrer 19 in rotation is of the magnetic type: more precisely, the mechanical stirrer 19 has magnetic or ferromagnetic parts configured and arranged so as to interact magnetically with permanent magnets distributed around the axis of a rotor 21 that can be driven in rotation by a motor 22. The rotor 21 is positioned below the station 2 of the receptacle 3 coaxially with the vertical tube 20.

The coffee machine can also have a heating means 4 and a frothing means 5 that are different from the ones illustrated thus far.

Therefore, irrespective of the specific construction of the heating means 4 and frothing means 5, the coffee machine 1 can advantageously have a spectrophotometer 23 configured to determine the content by weight of proteins and carbohydrates of the milk or plant-based beverage to be treated which is present in the receptacle 3.

The spectrophotometer 23 is based, in particular, on reflectance spectroscopy, which measures the spectral reflectance factor as a function of the wavelength of the incident radiation that is in the wavelength of the near infrared, visible and ultraviolet regions.

The coffee machine 1 has an electronic controller 24, to which the spectrophotometer 23, the heating means 4 and the frothing means 5 are connected.

Advantageously, the electronic controller 24 has in its memory a database containing associations Ak between combinations of values Sx, Sy of content by weight of proteins and carbohydrates of the milk or plant-based beverage to be treated and corresponding values Vi of at least one treatment parameter Pj for the milk or plant-based beverage to be treated.

Preferably, the coffee machine 1 also has a temperature sensor 25 for the determination, on contact or remotely, of the temperature of the milk or plant-based beverage to be treated which is present in the receptacle 3, for example an infrared temperature sensor outside the receptacle 3.

The temperature sensor 25 is also naturally connected to the electronic controller 24.

The result of the spectrophotometer 23, which can depend on the temperature of the milk or plant-based beverage, can thus be normalized by the temperature data of the milk or plant-based beverage detected by the temperature sensor 25.

The temperature sensor 25 and the spectrophotometer 23 can be integrated into a single device configured for an acquisition from a same region inside the receptacle 3.

In this manner, the accuracy with which the reading of the spectrophotometer 23 is normalized can be improved.

The database comprises a first association A1, wherein a first value V1 of at least one treatment parameter Pj for the milk or plant-based beverage corresponds to a content by weight of proteins no lower than a first threshold value S1 and a content by weight of carbohydrates no higher than a second threshold value S2.

The database further comprises a second association A2, wherein a second value V2 of at least one treatment parameter Pj for the milk or plant-based beverage corresponds to a content by weight of proteins lower than the first threshold value S1 and to a content by weight of carbohydrates higher than the second threshold value S2.

Finally, the database comprises a third association A3, wherein a third value V3 of at least one treatment parameter Pj for the milk or plant-based beverage corresponds to a content by weight of proteins higher than the first threshold value S1 and to a content by weight of carbohydrates higher than the second threshold value S2, or to a content by weight of proteins lower than the first threshold value S1 and to a content by weight of carbohydrates lower than the second threshold value S2.

The treatment parameters for the milk or plant-based beverage that can be adjusted based on the reading of the spectrophotometer 23 include the amount of thermal energy which the heating means 4 transfers overall and/or in the unit of time to the milk or plant-based beverage to be treated and/or the amount of air which the frothing means 5 transfers overall and/or in the unit of time to the milk or plant-based beverage to be treated.

It has been found to be convenient to set the first threshold value S1 between 0.1% and 0.5% by weight, preferably equal to 0.5% by weight, and the second threshold value S2 between 7% and 15% by weight, preferably equal to 7% by weight.

The treatment method thus comprises acquiring, in particular, in the case illustrated, determining by means of the spectrophotometer, the content by weight of proteins and carbohydrates of the milk or plant-based beverage to be treated and treating the milk or plant-based beverage with the value V1, V2, V3, of the treatment parameter Pj or parameters Pj corresponding, in the database, to the combination of the determined value Sx of the content by weight of proteins and the determined value Sy of the content by weight of carbohydrates.

Examples are provided below of adjustments that may be made with the coffee machine illustrated in FIG. 1 to produce hot frothed milk in a cup when the type of milk used changes.

Tables 1 and 2 below refer to the adjustment of treatment parameters for three types of milk indicated as #1, #2 and #3, made with the heating means 4 and the frothing means 5.

Table 1 refers to the adjustment of the value Vi of the temperature P1 of the steam flow entering the mixing chamber 13: with this adjustment one adjusts the amount of thermal energy to be transferred overall and in the unit of time to the milk to be treated.

TABLE 1

| | Sx | Sy | S1 | S2 | Vl(Pl) | V2(Pl) | V3(Pl) | Al | A2 | A3 |
|---|---|---|---|---|---|---|---|---|---|---|
| # 1 | 0.8% | 4% | 0.5% | 7% | 120° C. | | | x | | |
| # 2 | 0.3% | 10% | 0.5% | 7% | | 130° C. | | | x | |
| # 3 | 1.5% | 9% | 0.5% | 7% | | | 110° C. | | | x |

Table 2 refers to the adjustment of the value Vi of opening P2 (expressed as a % of the passage that is open relative to the maximum passage opening) of the valve 10: with this adjustment one adjusts the amount of air to be transferred overall and in the unit of time to the milk to be treated.

TABLE 2

| | Sx | Sy | S1 | S2 | Vl(P2) | V2(P2) | V3(P2) | Al | A2 | A3 |
|---|---|---|---|---|---|---|---|---|---|---|
| # 1 | 0.8% | 4% | 0.5% | 7% | 100% | | | x | | |
| # 2 | 0.3% | 10% | 0.5% | 7% | | 50% | | | x | |
| # 3 | 1.5% | 9% | 0.5% | 7% | | | 15% | | | x |

The adjustment can obviously regard other or different milk treatment parameters, for example the degree of moisture of the steam dispensed, the pressure of the steam dispensed, the duty cycle of the water pump that supplies the boiler for producing steam, etc.

Examples are provided below of adjustments that may be made with the coffee machine illustrated in FIG. 2 to produce hot frothed milk in a cup when the type of milk used changes.

Tables 3 and 4 below refer to the adjustment of treatment parameters for three types of milk indicated as #1, #2 and #3, made with the frothing means 5 and the heating means 4.

Table 3 refers to the adjustment of the value Vi of the rotation speed P3 (expressed in revolutions per minute) of the mechanical stirrer 19: with this adjustment one adjusts the amount of air to be transferred overall and in the unit of time to the milk to be treated.

TABLE 3

| | Sx | Sy | S1 | S2 | Vl(P3) | V2(P3) | V3(P3) | Al | A2 | A3 |
|---|---|---|---|---|---|---|---|---|---|---|
| # 1 | 0.8% | 4% | 0.5% | 7% | 600 rpm | | | x | | |
| # 2 | 0.3% | 10% | 0.5% | 7% | | 800 rpm | | | x | |
| # 3 | 1% | 9% | 0.5% | 7% | | | 300 rpm | | | x |

The adjustment can obviously regard other or different milk treatment parameters, for example the duty cycle of the air pump 17.

Table 4 refers to the adjustment of the value Vi of the activation time P4 (expressed in seconds) of the electric heater 7: with this adjustment one adjusts the amount of thermal energy to be transferred overall to the milk to be treated.

TABLE 4

| | Sx | Sy | S1 | S2 | VI(P4) | V2(P4) | V3(P4) | Al | A2 | A3 |
|---|---|---|---|---|---|---|---|---|---|---|
| # 1 | 0.8% | 4% | 0.5% | 7% | 110 sec | | | x | | |
| # 2 | 0.3% | 10% | 0.5% | 7% | | 130 sec | | | x | |
| # 3 | 1.5% | 9% | 0.5% | 7% | | | 120 sec | | | x |

As may be seen from the tables shown above, a first value V1 of the treatment parameters P1, P2, P3 and P4 for the milk is associated with the type of milk #1 that has a content by weight of proteins no lower than the first threshold value S1 and a content by weight of carbohydrates no higher than the second threshold value S2, a second value V2 of the treatment parameters P1, P2, P3 and P4 for the milk is associated with the type of milk #2 that has a content by weight of proteins lower than the first threshold value Si and a content by weight of carbohydrates higher than the second threshold value S2, and a third value V2 of the treatment parameters P1, P2, P3 and P4 for the milk is associated with the type of milk #3, which differs from both of the previous situations.

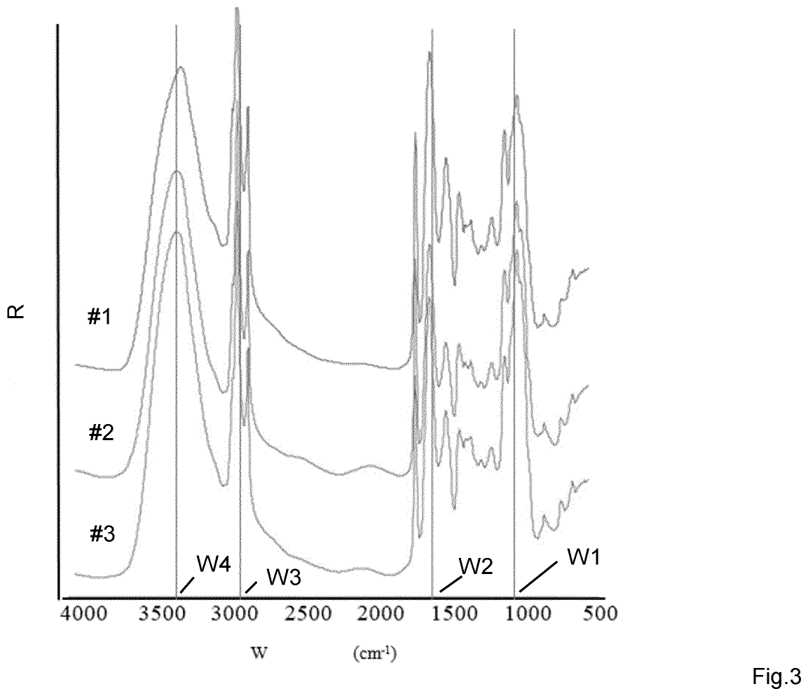

FIG. 3 graphically represents the data typically acquired by the spectrophotometer 23.

The wavenumber W is shown on the x-axis and the reflectance R is shown on the y-axis for the three types of milk #1, #2 and #3.

Peaks typically attributable to proteins, carbohydrates and fats can be noted: a first peak around the wavenumber W1=1000 cm⁻¹ related to the carbonyl group C═O of carbohydrates; a second peak around the wavenumber W2=1500 cm⁻¹ related to the carbonyl group C═O of carbohydrates and the amino group N—H of proteins; a third peak around the wavenumber W3=3000 cm⁻¹ related to the carbonyl group CH2 of fats; and a fourth peak around the wavenumber W4=3500 cm⁻¹ related to the amino group N—H of proteins.

Thanks to the method disclosed by the present invention, the result in the cup can be optimized irrespective of the type of milk or plant-based beverage used.

The method advantageously provides for an automatic selection, from the database, of the correct treatment parameters for the milk or plant-based beverage.

The thermal energy and air input to the milk or plant-based beverage to be treated is modulated based on the type of milk or plant-based beverage so as to always obtain the best result in terms of organoleptic properties, volume, consistency and visual appearance of the froth of the product dispensed into the cup.

The modulation of the thermal energy input is particularly relevant, since the characteristics of different types of milk or plant-based beverages can degrade at different temperatures.

The spectrophotometer 23 can also be used to determine the nature of the liquid present in the receptacle 3.

For example, if the spectrophotometer 23 identifies the presence of water in the receptacle 3, the electronic controller 24 can automatically activate the performance of a cleaning treatment, which may be displayed, for example, on an interface of the coffee machine 1.

It should be noted that the treatment parameters which can be adjusted and the way in which they can be adjusted can be multiple and different from the ones illustrated.

Preferably, continual experimentation also on new commercially available types of milk and plant-based beverages can be provided for in order to update the database and reprogram the memory of the electronic controller with the updated database.

Finally, as mentioned, the method is applicable to any device, not necessarily a coffee machine, which has an electronic controller with a programmable memory, a heating means and a frothing means adjustable by the electronic controller, and optionally a spectrophotometer suitable for determining the content by weight of proteins and carbohydrates of the milk or plant-based beverage to be treated and communicating the data thus determined to the electronic controller for the purpose of adjusting the heating means and/or the frothing means.

The method for treating frothed milk thus conceived is susceptible of numerous modifications and variants, all falling within the scope of the inventive concept; moreover, all the details may be replaced by technically equivalent elements.

The scope of protection obviously extends to the treatment not only of milk, but also, as is evident from what was described above, of plant-based beverages.

The step of acquiring the content by weight of proteins and carbohydrates of the milk or plant-based beverage to be treated need not necessarily be carried out by means of a spectrophotometer.

In fact, the acquisition step can include an automatic optical reading of information shown on a package of the milk or plant-based beverage to be treated.

In such a case, the optical reading can be carried out by a mobile electronic device equipped with a camera and software for automatic recognition of information relating to the content by weight of proteins and carbohydrates of the milk or plant-based beverage to be treated, and the mobile electronic device can be connected by means of a wireless connection, for example a WiFi or Bluetooth connection, to a coffee machine or a milk frothing device that performs the treatment. In another case, the acquisition step can include a personal information acquisition step.

In such a case, the acquisition can comprise a step in which information relating to the content by weight of proteins and carbohydrates of the milk or plant-based beverage to be treated is manually uploaded by the user into the memory of the controller of the treatment device, either via a physical interface of the treatment device or by means of a mobile electronic device having a wireless connection, for example a WiFi or Bluetooth connection, to the treatment device.

The choice of the threshold values S1 and S2 can also be different from the one illustrated above, and in particular in some applications a first threshold value S1 higher than 1.5% by weight and a second threshold value S2 higher than 7% by weight can be set.

The materials used, as well as the dimensions, may in practice be any whatsoever according to needs and the state of the art.

1. A method for treating milk or a plant-based beverage,
creating a database containing associations between combinations of values of content by weight of proteins and carbohydrates of the milk or plant-based beverage to be treated and corresponding values of at least one treatment parameter for the milk or plant-based beverage;
acquiring the content by weight of proteins and carbohydrates of the milk or plant-based beverage to be treated; and
treating the milk or plant-based beverage with the value of said at least one treatment parameter corresponding in the database to the acquired values of the content by weight of proteins and carbohydrates.

2. The method for treating milk or a plant-based beverage according to claim 1, wherein said acquisition step includes a spectrophotometric determination performed directly on said milk or plant-based beverage to be treated.

3. The method for treating milk or a plant-based beverage according to claim 1, wherein said acquisition step includes an automatic optical reading of information provided on a package of said milk or plant-based beverage to be treated.

4. The method for treating milk or a plant-based beverage according to claim 1, wherein said acquisition step includes a personal acquisition of information.

5. The method for treating milk according to claim 1, wherein said database comprises a first association, wherein a first value of said at least one treatment parameter corresponds to a content by weight of proteins no lower than a first threshold value and a content by weight of carbohydrates no higher than a second threshold value.

6. The method for treating milk according to claim 5, wherein database comprises a second association, wherein a second value of said at least one treatment parameter corresponds to a content by weight of proteins lower than said first threshold value and to a content by weight of carbohydrates higher than said second threshold value.

7. The method for treating milk according to claim 7, wherein said database comprises a third association, wherein a third value of said at least one treatment parameter corresponds to a content by weight of proteins higher than said first threshold value and to a content by weight of carbohydrates higher than said second threshold value, or to a content by weight of proteins lower than said first threshold value and to a content by weight of carbohydrates lower than said second threshold value.

8. The method for treating milk according to claim 5, wherein said first threshold value is comprised between 0.1% and 0.5% by weight and said second threshold value is comprised between 7% and 15% by weight.

9. The method for treating milk according to claim 8, wherein said first threshold value is equal to 0.5% by weight and said second threshold value is equal to 7% by weight.

10. The method for treating milk according to claim 1, wherein said at least one treatment parameter comprises at least the amount of thermal energy to be transferred overall and/or in the unit of time to the milk or plant-based beverage to be treated.

11. The method for treating milk according to claim 1, wherein said at least one treatment parameter comprises at least an amount of air to be transferred overall and/or in the unit of time to the milk or plant-based beverage to be treated.

12. A device, particularly a coffee machine or a milk frothing device, comprising a receptacle for a milk or plant-based beverage to be treated, a station for said receptacle, a means for heating the milk, and a means for frothing the milk or plant-based beverage, a spectrophotometer configured to determine the content by weight of proteins and carbohydrates of the milk or plant-based beverage to be treated present in said receptacle, and in that it has an electronic controller having in its memory a database containing associations between combinations of values of content by weight of proteins and carbohydrates of the milk or plant-based beverage to be treated and corresponding values of at least one treatment parameter for the milk or plant-based beverage to be treated, said controller being configured to control the treatment of the milk or plant-based beverage with the value of said at least one corresponding treatment parameter in the database at the determined values of the content by weight of proteins and carbohydrates.

13. The device according to claim 12, wherein at least one treatment parameter includes at least the amount of thermal energy to be transferred overall and/or in the unit of time to the milk or plant-based beverage to be treated.

14. The device according to claim 12, wherein said at least one treatment parameter further includes at least an amount of air to be transferred overall and/or in the unit of time to the milk or plant-based beverage to be treated.

15. The device according to claim 14, further comprising a temperature sensor for the determination, on contact or remotely, of the temperature of the milk or plant-based beverage to be treated present in said receptacle.
